(19) United States
(12) Patent Application Publication
(10) Pub. No.: US 2024/0124520 A1
(43) Pub. Date: Apr. 18, 2024

(54) N-ACYLATED HISTIDINE DIPEPTIDES AS ANTICANCER AGENTS

(71) Applicant: THE BOARD OF TRUSTEES OF THE LELAND STANFORD JUNIOR UNIVERSITY, Stanford, CA (US)

(72) Inventors: Michael BRUNO, Stanford, CA (US); Jijuan GU URBAN, Stanford, CA (US); Jennifer LI-POOK-THAN, Stanford, CA (US); Teri SLIFER, Stanford, CA (US); Michael SNYDER, Stanford, CA (US)

(73) Assignee: THE BOARD OF TRUSTEES OF THE LELAND STANFORD JUNIOR UNIVERSITY, Stanford, CA (US)

(21) Appl. No.: 17/769,169

(22) PCT Filed: Oct. 10, 2020
(86) PCT No.: PCT/US2020/055707

§ 371 Date: Apr. 14, 2022

Related U.S. Application Data
(60) Provisional application No. 62/916,001, filed on Oct. 16, 2019.

(51) Int. Cl. C07K 5/078; A61P 35/00

(52) U.S. Cl. CPC C07K 5/06147; A61P 35/00; A61K 38/00

(57) ABSTRACT

N-Acylated histidine dipeptides of formula

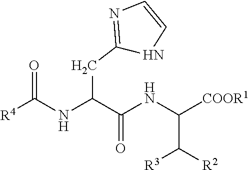

are disclosed. The compounds are useful for treating breast cancer.

### CROSS-REFERENCE TO RELATED APPLICATIONS

This application claims benefit of priority to U.S. Provisional Patent Application No. 62/916,001, filed Oct. 16, 2019, the entire contents of which are incorporated herein by reference.

### FEDERALLY SPONSORED RESEARCH

This invention was made with Government support under contracts GM062480 and HG007735 awarded by the National Institutes of Health. The Government has certain rights in the invention.

### FIELD

This disclosure relates to N-acylated histidine dipeptides and derivatives thereof useful in the treatment of cancer, particularly breast cancer.

### BACKGROUND

Despite its high survival rate, breast cancer is still the most common cancer affecting women. In the US alone, there are 271,270 new breast cancer cases estimated for 2019 and 42,260 estimated deaths for the same year from this disease. Breast cancer treatment varies depending on factors such as tumor stage, size, and patient preference. While the majority of treatment involves breast-conserving surgery or mastectomy accompanied by radiotherapy, in approximately 35% of early stage (I and II) cancers the treatment will include a drug therapy (targeted and/or untargeted). In stage III and IV breast cancers, at least 58% are treated with drug therapy in combination with surgery and/or radiotherapy. Small molecule therapies for treating breast cancer that exhibit greater efficacy and/or lower toxicity are needed.

### SUMMARY

In one aspect, the disclosure relates to A compound of formula I.

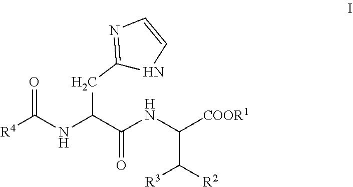

wherein
R¹ is H or (C₁-C₆)alkyl;
R² is (C₁-C₁₀)hydrocarbyl;
R³ is (C₁-C₆)alkyl;
R⁴ is chosen from (C₁₄-C₂₀)hydrocarbyl; (C₁₄-C₂₀)hydrocarbyl substituted with one or more of halogen, hydroxy, mercapto, (C₁-C₆)acyl, (C₁-C₆)alkoxy, (C₁-C₆)haloalkoxy, amino, (C₁-C₆)alkylamino, di(C₁-C₆)alkylamino, and (C₁-C₆)alkylthio; (C₁₀-C₂₀)oxaalkyl; (C₁₀-C₂₀)thiaalkyl and (C₁₀-C₂₀)azaalkyl.

In another aspect, the disclosure relates to pharmaceutical compositions comprising a pharmaceutically acceptable carrier and a compound of formula I.

In another aspect, the disclosure relates to method of treating breast cancer comprising administering to a mammal in need of treatment a therapeutically effective amount of a compound of formula I.

In another aspect, the disclosure relates to a compound of Formula I for use in treatment of breast cancer.

In another aspect, the disclosure relates to a use of a compound of Formula I in the manufacture of a medicament for treating breast cancer.

### BRIEF DESCRIPTION OF THE DRAWINGS

These and other features, aspects, and advantages of the present disclosure will become better understood when the following detailed description is read with reference to the accompanying drawings, wherein:

FIG. 1: Dose Response curve for MBC017 with MDA-MB-231 cells. Compound MBC017 was incubated with MDA-MB-231 triple-negative human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart. The data were not sufficient to calculate an accurate IC50 value.

FIG. 2: Dose Response curve for MBC0171 with MDA-MB-231 cells. Compound MBC0171 was incubated with MDA-MB-231 triple-negative human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 3: Dose Response curve for MBC0172 with MDA-MB-231 cells. Compound MBC0172 was incubated with MDA-MB-231 triple-negative human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 4: Dose Response curve for MBC0174 with MDA-MB-231 cells. Compound MBC0174 was incubated with MDA-MB-231 triple-negative human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 5: Dose Response curve for MBC0179 with MDA-MB-231 cells. Compound MBC0179 was incubated with MDA-MB-231 triple-negative human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 6: Dose Response curve for MBC0179 with hTERT-HME1 cells in MEGM Media. Compound MBC0179 was incubated with hTERT-HIE1 healthy human breast epithelial cells in growth factor-rich MEGM media at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 7: Dose Response curve for MBC0179 with MDA-MB-231 cells in MEGM Media. Compound MBC0179 was incubated with MDA-MB-231 triple-negative human breast cancer cells in growth factor-rich MEGM media at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 8: Dose Response curve for MBC0179 with MCF7 cells. Compound MBC0179 was incubated with MCF7, estrogen receptor positive, human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 9: Dose Response curve for MBC0179 with MDA-MB-468 cells. Compound MBC0179 was incubated with MDA-MB-468 triple-negative human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 10: Dose Response curve for MBC0179 with ZR-75-30 cells. Compound MBC0179 was incubated with ZR-75-30 human breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 11: Dose Response curve for MBC0179 with K-562 cells. Compound MBC0179 was incubated with K-562 human chronic myelogenous leukemia cells, a non-adherent cell line, at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 12: Dose Response curve for MBC0179 with HEPG2 cells. Compound MBC0179 was incubated with HEPG2 human liver carcinoma cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 13: Dose Response curve for MBC0179 with A549 cells. Compound MBC0179 was incubated with A549 human lung cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 14: Dose Response curve for MBC0179 with OVCAR-3 cells. Compound MBC0179 was incubated with OVCAR-3 human ovarian cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 15: Dose Response curve for MBC0179 with SK—N—SH cells. Compound MBC0179 was incubated with SK—N—SH human neuroblastoma cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 16: Dose Response curve for MBC0179 with 4T1 cells. Compound MBC0179 was incubated with 4T1 mouse triple-negative breast cancer cells at the indicated concentrations for 3 days at 37° C. prior to Cell Titer Blue fluorescence-based cell viability assay. Normalized cell viability data, expressed as a percentage, are averaged from three biological replicates and standard error represented by error bars for each averaged data point (empty squares). The compound concentrations are represented on log scale on the x-axis in μM units. Data were analyzed using free software called DrFit, as described in Nature Scientific Reports, Article number: 14701, (2015). DrFit was used to fit the plotted data points to the Hill Equation (red curve) and produce an IC50 value (μM), displayed on the chart.

FIG. 17: 4T1 Mouse Breast Cancer Lung Metastasis Study with MBC0179. Compound MBC0179 (in 7% w/v Cremophore EL/10% v/v ethanol/90% v/v saline vehicle) or vehicle only was administered intravenously to three groups of six Balb/c mice (8 week old) carrying 4T1 murine breast cancer cell line-derived mammary tumors, one week post-implantation. After twice weekly dosing for four weeks with vehicle (blue bar), 10 mg/kg (red bar) or 15 mg/kg (green bar) MBC0179, the mice were humanely euthanized according to IACUC guidelines and lungs excised for 4T1 colonogenic assay in order to provide a measure of 4T1 metastasis to the lungs. The colony counts were plotted, averaged and standard error calculated (error bars). A two-tailed t-test of statistical significance was performed to compare the vehicle and 15 mg/kg MBC0179 treatment groups, indicated on chart with p-value. Additonally, the percentage reduction in lung-derived 4T1 colonies between the vehicle and 15 mg/kg MBC0179 treatment group was calculated based on the average colony counts for each group, normalizing to the vehicle group, indicated on chart.

### DETAILED DESCRIPTION

The compounds described herein inhibit tumor growth. The compounds that are useful in the methods described herein fall into a genus of formula I:

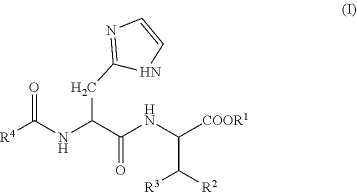

In these compounds, R¹ can be H or (C₁-C₆)alkyl. In some embodiments, R¹ is hydrogen; in some embodiments, R¹ is methyl or ethyl.

R² is (C₁-C₁₀)hydrocarbyl, and R³ is (C₁-C₆)alkyl. In some embodiments, R² is ethyl and R³ is methyl. In other embodiments, R² and R³ are both methyl.

R⁴ is chosen from (C₁₄-C₂₀)hydrocarbyl; (C₁₄-C₂₀)hydrocarbyl substituted with one or more of halogen, hydroxy, mercapto, (C₁-C₆)acyl, (C₁-C₆)alkoxy, (C₁-C₆)haloalkoxy, amino, (C₁-C₆)alkylamino, di(C₁-C₆)alkylamino, and (C₁-C₆)alkylthio; (C₁₀-C₂₀)oxaalkyl; (C₁₀-C₂₀)thiaalkyl and (C₁₀-C₂₀)azaalkyl. In some embodiments, R⁴ is a (C₁₄-C₂₀)aliphatic hydrocarbon. In some embodiments, R⁴ is a fluorinated (C₁₄-C₂₀)aliphatic hydrocarbon. In some embodiments, R⁴ is a linear (C₁₄-C₂₀)alkyl. In some embodiments, R¹ is H and R⁴ is (C₁₄-C₂₀)alkyl.

The disclosed compounds are basic when R¹ is alkyl and amphoteric when R¹ is hydrogen. The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids. Suitable pharmaceutically acceptable acid addition salts for the compounds of the present invention include acetic, adipic, alginic, ascorbic, aspartic, benzenesulfonic (besylate), benzoic, boric, butyric, camphoric, camphorsulfonic, carbonic, citric, ethanedisulfonic, ethanesulfonic, ethylenediaminetetraacetic, formic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, oleic, pamoic, pantothenic, phosphoric, pivalic, polygalacturonic, salicylic, stearic, succinic, sulfuric, tannic, tartaric acid, teoclatic, p-toluenesulfonic, and the like. When R¹ is not alkyl, in addition to internal salts, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine. Further pharmaceutically acceptable salts include, when appropriate, nontoxic ammonium cations and carboxylate, sulfonate and phosphonate anions attached to alkyl having from 1 to 20 carbon atoms. Although pharmaceutically acceptable counter ions will be preferred for preparing pharmaceutical formulations and for use in therapeutic methods, other anions are quite acceptable as synthetic intermediates. Thus the anion may be pharmaceutically undesirable when such salts are chemical intermediates.

Throughout this specification the terms and substituents retain their definitions.

Hydrocarbon refers to scaffolds containing only hydrogen and carbon. It includes alkyl, cycloalkyl, polycycloalkyl, alkenyl, alkynyl, aryl and combinations thereof. Examples include phenyl, benzyl, phenethyl, cyclohexylmethyl, camphoryl, 9-hexadecenyl, 6-octadecenyl, 9-octadecenyl, 9-eicosenyl, and 13-docosenyl.

Alkoxy or alkoxyl refers to groups of from 1 to 6 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like.

Oxaalkyl refers to alkyl residues in which one or more carbons (and their associated hydrogens) have been replaced by oxygen. Examples include 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively.

Acyl refers to groups of 1, 2, 3, 4, and 5 carbon atoms of a straight, branched, cyclic configuration, saturated, unsaturated and aromatic and combinations thereof, attached to the parent structure through a carbonyl functionality. Examples include formyl, acetyl, benzoyl, propionyl, isobutyryl and the like.

Aryl and heteroaryl mean a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S.

As used herein, the term “optionally substituted” may be used interchangeably with “unsubstituted or substituted”. The term “substituted” refers to the replacement of one or more hydrogen atoms in a specified group with a specified radical. For example, substituted (C₁₄-C₂₀)hydrocarbyl refers to a (C₁₄-C₂₀)hydrocarbyl residue wherein one or more H atoms are replaced with halogen, hydroxy, mercapto, (C₁-C₆)acyl, (C₁-C₆)alkoxy, (C₁-C₆)haloalkoxy, amino, (C₁-C₆)alkylamino, di(C₁-C₆)alkylamino, or (C₁-C₆)alkylthio. An example of such a substituted hydrocarbyl residue would be ricinoleyl (i.e. 12-hydroxy-9-octadecenyl). Although in most cases of “optionally substituted” residues, 1, 2 or 3 hydrogen atoms are replaced with a specified radical, in the case of fluorohydrocarbon residues, more than three hydrogen atoms can be replaced by fluorine; indeed, all available hydrogen atoms could be replaced by fluorine, e.g. perfluoropropyl.

The compounds described herein contain at least two asymmetric centers and thus give rise to enantiomers, diastereomers, and other stereoisomeric forms that may be defined, in terms of absolute stereochemistry, as (R)- or (S)-. The present disclosure is meant to include all such possible isomers, as well as their racemic and optically pure forms. The (S,S) diastereomer of the dipeptide is preferred, i.e. the dipeptide comprising two L-amino acids. Optically active (R)- and (S)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

In some examples, a portion of a compound of Formula I may comprise an amino acid. Amino acids may exist as different stereoisomers, referred to conventionally by those skilled in the relevant field as D- and L-isomers. L-isomers of amino acids are those produced and incorporated into proteins by living cells. In some compounds of Formula I, comprising in part one or two amino acids, each independently or both amino acid components may exist as an L-isomer exclusively or predominantly (greater than 90% w/v), or a D-isomer exclusively or predominantly. In other examples, each independently or both amino acid components may exist in roughly equal proportions of an L- and D-isoform. In some examples, one isoform is predominantly in an L- form or a D-form, while the other exists as a mixture of both isoforms. In other examples, both amino acid components are in D- or in L-form, while in other one may be in a D-form while the other is in an L-form. Without being limited to a particular mechanism of action or degree or duration of effectiveness of a compound, whereas naturally occurring polypeptides in cells are comprised predominantly of amino acids with an L-configuration, as disclosed herein compounds of Formula I including an amino acid component or components in a D-form may be more resistant to peptidase action than compounds having amino acid components that are not in D-form and therefore exhibit prolonged or enhanced physiological efficacy upon administration to a cell or organism.

As used herein, and as would be understood by the person of skill in the art, the recitation of “a compound”—unless expressly further limited—is intended to include salts of that compound. In a particular embodiment, the term “compound of formula I” refers to the pharmaceutically acceptable salt.

It will be recognized that the compounds of this disclosure can exist in radiolabeled form, i.e., the compounds may contain one or more atoms containing an atomic mass or mass number different from the atomic mass or mass number usually found in nature. Alternatively, a plurality of molecules of a single structure may include at least one atom that occurs in an isotopic ratio that is different from the isotopic ratio found in nature. Radioisotopes of hydrogen, carbon, phosphorous, fluorine, chlorine and iodine include, for example, ²H, ³H, ¹¹C, ³C, ⁴C, ⁵N, ³⁵S, ¹⁸F, ³⁶Cl, ¹²³I, ¹²⁵I, ¹³¹I and ¹³³I. Compounds that contain those radioisotopes and/or other radioisotopes of other atoms are within the scope of this disclosure. Compounds containing ³H, ¹⁴C and iodine radioisotopes are particularly preferred for their ease in preparation and detectability. Compounds that contain isotopes ¹¹C, ¹³N, ¹⁵O and ¹⁸F are well suited for positron emission tomography. Radiolabeled compounds of formulae I and II of this disclosure can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

Although this disclosure is susceptible to embodiment in many different forms, preferred embodiments of the disclosure are shown. It should be understood, however, that the present disclosure is to be considered as an exemplification of the principles of this disclosure and is not intended to limit the disclosure to the embodiments illustrated.

In a further composition aspect, the disclosure relates to a pharmaceutically acceptable carrier and a compound of formula I. Such pharmaceutical compositions may additionally comprise other anti-tumor agents and excipients. As they are intended for treatment of cancer and not for forming surfactants or complexes, they will generally be free of complexing metals, such as copper. Preferred carriers will be aqueous and free of water-immiscible solvents. The carrier(s) must be “acceptable” in the sense of being compatible with the other ingredients of the formulation and not deleterious to the recipient thereof. The compositions may be formulated for oral, topical or parenteral administration. For example, they may be given intravenously, intraarterially, subcutaneously, and directly into the CNS—either intrathecally or intracerebroventricularly.

Formulations include those suitable for oral, parenteral (including subcutaneous, intradermal, intramuscular, intravenous and intraarticular), rectal and topical (including dermal, buccal, sublingual and intraocular) administration. The compounds are preferably administered orally or by injection (intravenous or subcutaneous). The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity. The formulations may conveniently be presented in unit dosage form and may be prepared by any of the methods well known in the art of pharmacy. In general, the formulations are prepared by uniformly and intimately bringing into association the active ingredient with liquid carriers or finely divided solid carriers or both and then, if necessary, shaping the product into the desired formulation.

Formulations of the present disclosure suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose or multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described. Preferred unit dosage formulations are those containing an effective dose, or an appropriate fraction thereof, of the active ingredient.

It should be understood that in addition to the ingredients particularly mentioned above, the formulations of this disclosure may include other agents conventional in the art having regard to the type of formulation in question, for example those suitable for oral administration may include flavoring agents.

As used herein, the terms “treatment” or “treating,” or “palliating” or “ameliorating” refer to an approach for obtaining beneficial or desired results including but not limited to therapeutic benefit and/or a prophylactic benefit. By therapeutic benefit is meant amelioration of the underlying disorder being treated. Also, a therapeutic benefit is achieved with the amelioration of one or more of the physiological symptoms associated with the underlying disorder such that an improvement is observed in the patient, notwithstanding that the patient may still be afflicted with the underlying disorder. In other examples, a compound of Formula I may be administered to an animal or human subject, or to a sample (such as cell culture or other in vitro system) for purposes other than treatment. Another example includes a compound of Formula I for use in treatment of breast cancer. Yet another example includes a use of a compound of Formula I in the manufacture of a medicament for treating breast cancer. In some examples, a single compound of Formula I, or any combinations of two or more compounds of Formula I, may be administered, for treatment or otherwise.

The compounds may be synthesized by methods well-known in the art. For example, histidine can be substituted for alanine in the synthesis described in US published application 2012/0308646 in paragraph [0179] et seq. In like manner other compounds of formula I may be synthesized utilizing other amino acids having the —CHR²R³ side chain.

Some examples of compounds of Formula I that have been tested include those identified in Table 1, as follows: Full chemical names are given for each molecule tested with the corresponding compound and peptide name IDs in each row. The cell line against which each compound was tested and the resultant IC50 value (μM) are displayed next to the figure number that displays the dose response data used to calculate the IC50 values. In the cell line column, all cells were shown as tested in DMEM or RPMI media, indicated in parenthesis, and as described in the main text. However, in some cases MEGM media was used for a comparison of MBC0179 peptide with hTERT-HMIE1 and MIDA-MB-231 cells in order to minimize differences due to incubation in growth factor-rich MEGM media and facilitate a true comparison. In the case of MBIC017, the compound did reduce cell viability but not enough to enable an accurate calculation of the IC50, and is shown as not calculated (NC).

TABLE 1
Summary of Results for Cell Titer Blue In Vitro Cell Viability Assays.

| Compound | Peptide | | | IC₅₀ | Figure |
|---|---|---|---|---|---|
| # | ID | Peptide Description | Cell Line Tested | (μM) | # |
| 1 | MBC017 | N-[N-(1-oxohexadecyl)-L- | MDA-MB-231 (DMEM) | NO | 1 |
| | | histidyl]-L-valine | | | |
| 2 | MBC0171 | N-[N-(1-oxohexadecyl)-L- | MDA-MB-231 (DMEM) | 6730 | 2 |
| | | histidyl]-L-isoleucine | | | |
| 3 | MBC0172 | N-[N-(1-oxohexadecyl)-L- | MDA-MB-231 (DMEM) | 13.1 | 3 |
| | | histidyl]-L-valine ethyl | | | |
| | | ester | | | |
| 4 | MBC0174 | N-[N-(1-oxohexadecyl)- | MDA-MB-231 (DMEM) | 62.1 | 4 |
| | | D-histidyl]-D-valine | | | |
| 5 | MBC0179 | N-[N-(1-oxohexadecyl)- | MDA-MB-231 (DMEM) | 14.7 | 5 |
| | | D-histidyl]-D-valine ethyl | | | |
| | | ester | | | |
| 5 | MBC0179 | N-[N-(1-oxohexadecyl)- | hTERT-HME1 (MEGM) | 270 | 6 |
| | | D-histidyl]-D-valine ethyl | | | |
| | | ester | | | |
| 5 | MBC0179 | N-[N-(1-oxohexadecyl)- | MDA-MB-231 (MEGM) | 8.88 | 7 |
| | | D-histidyl]-D-valine ethyl | | | |
| | | ester | | | |
| 5 | MBC0179 | N-[N-(1-oxohexadecyl)- | MCF7 (DMEM) | 355 | 8 |
| | | D-histidyl]-D-valine ethyl | | | |
| | | ester | | | |
| 5 | MBC0179 | N-[N-(1-oxohexadecyl)- | MDA-MB-468 (DMEM) | 15.7 | 9 |
| | | D-histidyl]-D-valine ethyl | | | |
| | | ester | | | |
| 5 | MBC0179 | N-[N-(1-oxohexadecyl)- | ZR-75-30 (DMEM) | 9.7 | 10 |
| | | D-histidyl]-D-valine ethyl | | | |
| | | ester | | | |
| 5 | MBC0179 | N-[N-(1-oxohexadecyl)- | K562 (DMEM) | 6.11 | 11 |
| | | D-histidyl]-D-valine ethyl | | | |
| | | ester | | | |
| 5 | MBC0179 | N-[N-(1-oxohexadecyl)- | HEPG2 (DMEM) | 24.9 | 12 |
| | | D-histidyl]-D-valine ethyl | | | |
| | | ester | | | |
| 5 | MBC0179 | N-[N-(1-oxohexadecyl)- | A549 (DMEM) | 10.5 | 13 |
| | | D-histidyl]-D-valine ethyl | | | |
| | | ester | | | |
| 5 | MBC0179 | N-[N-(1-oxohexadecyl)- | OVCAR-3 (RPMI) | 9.22 | 14 |
| | | D-histidyl]-D-valine ethyl | | | |
| | | ester | | | |
| 5 | MBC0179 | N-[N-(1-oxohexadecyl)- | SK-N-SH (DMEM) | 35 | 15 |
| | | D-histidyl]-D-valine ethyl | | | |
| | | ester | | | |
| 5 | MBC0179 | N-[N-(1-oxohexadecyl)- | 4T1 (RPMI) | 25.5 | 16 |
| | | D-histidyl]-D-valine ethyl | | | |
| | | ester | | | |

Assays and Test Results

A commercially available in vitro cell viability reagent (Cell Titer Blue) was employed to determine the anti-cancer effectiveness of several compounds (1-5) against multiple cancer and healthy tissue-derived cell lines. Cell lines are summarized in Table 2, as follows: All cell lines and information on each cell lines origins and characteristics were procured from ATCC (www.atcc.org). Common names and ATCC product IDs are shown in the first (leftmost) column. From the left, the second and third columns describe the organism and tissue from which each cell line was isolated. The fourth and fifth columns indicate the cell type and disease status (e.g. normal versus carcinoma) of each cell line. The sixth column provides a summary of what compounds were tested against each cell line using an in vitro cell viability assay. Peptide name IDs are shown in parenthesis for each compound, numbered 1 through 5.

TABLE 2
Descriptions of Cell Lines Tested.

| ATCC Cell | | | | | Tested with |
|---|---|---|---|---|---|
| Line | Organism | Tissue | Cell Type | Disease/Normal | Compound # |
| MDA-MB-231 | Homo | mammary | epithelial | adenocarcinoma | 1 (MBC017) |
| (ATCC ® | sapiens, | gland/breast; | | | 2 (MBC0171) |
| HTB-26 ™) | human | derived from | | | 3 (MBC0172) |
| | | metastatic site: | | | 4 (MBC0174) |
| | | pleural effusion | | | 5 (MBC0179) |
| MCF7 | Homo | mammary | epithelial | adenocarcinoma | 5 (MBC0179) |
| (ATCC ® | sapiens, | gland, breast; | | | |
| HTB-22 ™) | human | derived from | | | |
| | | metastatic site: | | | |
| | | pleural effusion | | | |
| MDA-MB-468 | Homo | mammary | epithelial | adenocarcinoma | 5 (MBC0179) |
| (ATCC ® | sapiens, | gland/breast; | | | |
| HTB-132 ™) | human | derived from | | | |
| | | metastatic site: | | | |
| | | pleural effusion | | | |
| ZR-75-30 | Homo | mammary | epithelial | ductal carcinoma | 5 (MBC0179) |
| (ATCC ® | sapiens, | gland/breast; | | | |
| CRL-1504 ™) | human | derived from | | | |
| | | metastatic site: | | | |
| | | ascites | | | |
| hTERT-HME1 | Homo | Breast; | Epithelial- | normal | 5 (MBC0179) |
| (ATCC ® | sapiens, | mammary | like | | |
| CRL-4010 ™) | human | gland; | | | |
| | | Epithelium | | | |
| K-562 | Homo | bone marrow | lympho- | chronic | 5 (MBC0179) |
| (ATCC ® | sapiens, | | blast | myelogenous | |
| CCL-243 ™) | human | | | leukemia (CML) | |
| Hep G2 | Homo | liver | epithelial | hepatocellular | 5 (MBC0179) |
| [HEPG2] | sapiens, | | | carcinoma | |
| (ATCC ® | human | | | | |
| HB-8065 ™) | | | | | |
| A549 | Homo | lung | epithelial | Carcinoma | 5 (MBC0179) |
| (ATCC ® | sapiens, | | cell (KRAS | | |
| CRM-CCL-185 ™) | human | | CRM) | | |
| OVCAR-3 | Homo | ovary | epithelial | adenocarcinoma | 5 (MBC0179) |
| [OVCAR3] | sapiens, | | | | |
| (ATCC ® | human | | | | |
| HTB-161 ™) | | | | | |
| SK-N-SH | Homo | brain; derived | epithelial | neuroblastoma | 5 (MBC0179) |
| (ATCC ® | sapiens, | from metastatic | | | |
| HTB-11 ™) | human | site: bone | | | |
| | | marrow | | | |
| 4T1 | Mus | mammary gland | epithelial | This tumor is an | 5 (MBC0179) |
| (ATCC ® | musculus, | | | animal stage IV | |
| CRL-2539 ™) | mouse | | | breast cancer. | |

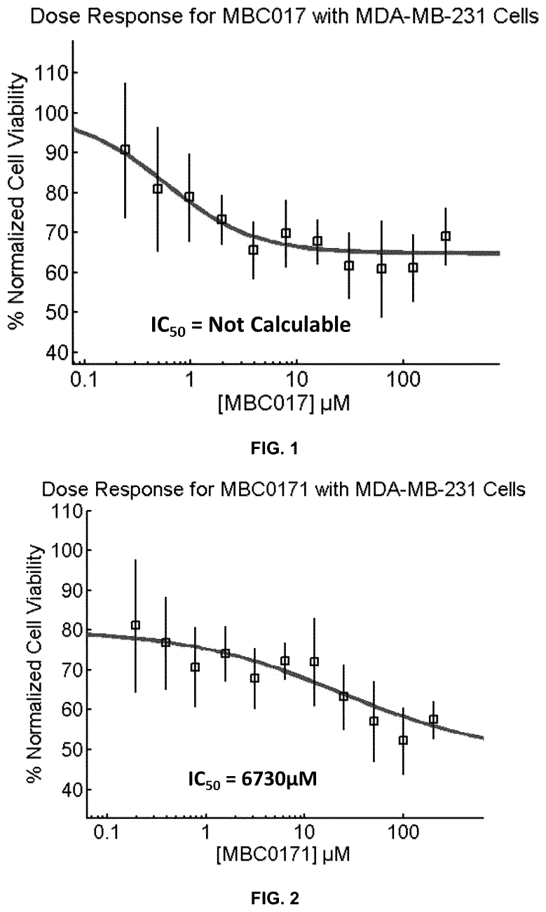

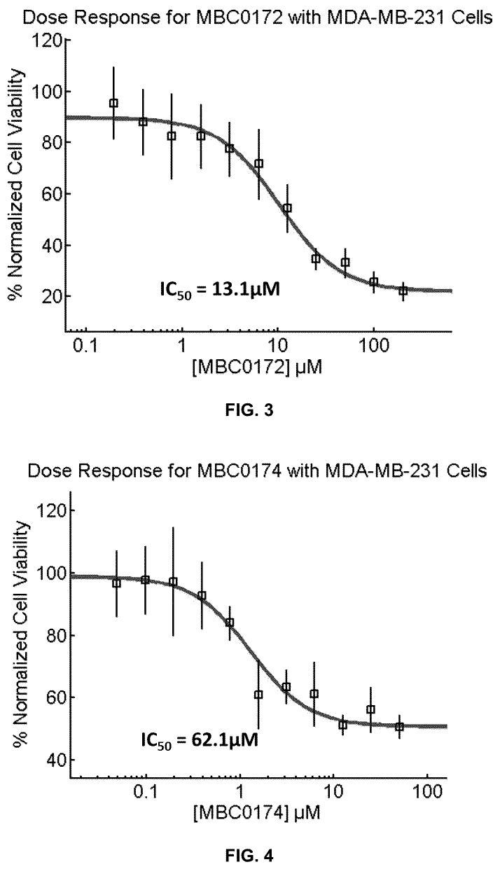

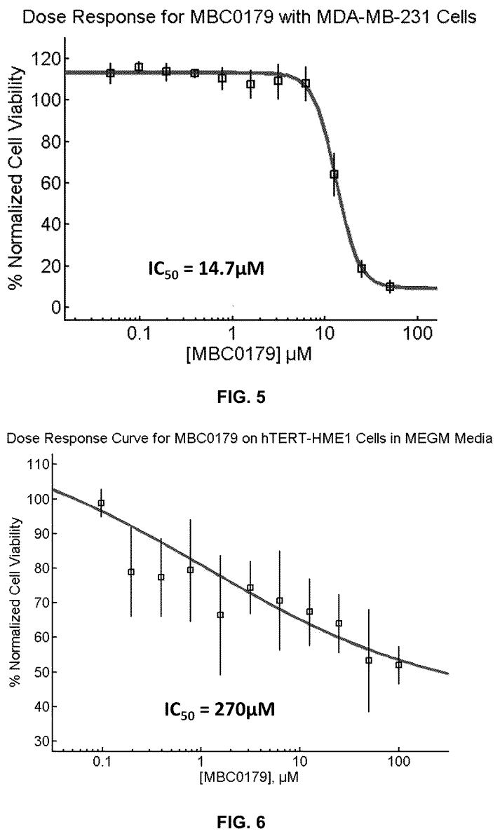

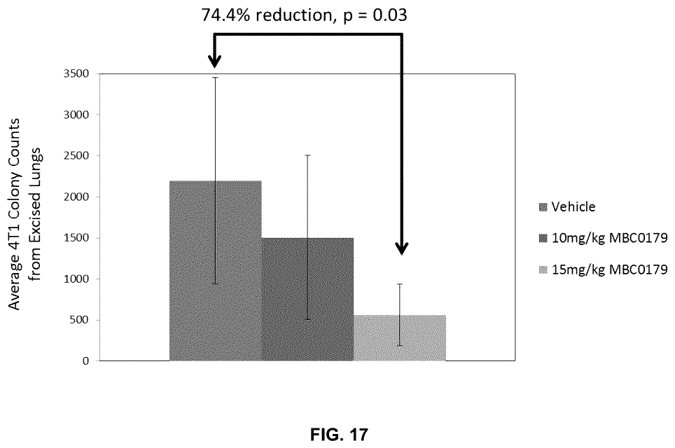

The Cell Titer Blue reagent (available from Promega, Inc.) is a buffered solution containing resazurin, a compound that is converted to resorufin, a detectable fluorescent product, by metabolically active cells. The fluorescence intensity of resorufin product therefore serves as a proportional measure of the number of viable cells, permitting changes in cell viability within cancer or other cell lines to be monitored in response to the addition of cytotoxic or cytostatic agents. Various human breast tissue-derived cell lines are widely employed to study the effects of potential therapeutics against breast cancer in vitro. Human breast cancer cell lines may be subdivided into groups based upon hormone receptor status, i.e. estrogen, progesterone and human epidermal growth factor positive or negative. A healthy human breast tissue-derived cell line, hTERT-HME1 may be used to determine the effect of therapeutic agents on healthy human breast tissue in vitro. Additionally, a highly metastatic and aggressive murine (mouse) breast cancer cell line, 4T1, has been extensively used to monitor breast cancer metastasis from the mammary fat pads of mice to the lungs in order to systematically survey the anti-metastatic effectiveness of anti-cancer therapeutics in an in vivo animal model. Metastases to the lungs may be conveniently quantified by a well-established clonogenic assay. Other human tissue-derived model cell lines, representative of various cancers, other than breast cancer, may be utilized to assess the potential of anti-cancer compounds in vitro. These include, K-562 (chronic myelogenous leukemia), HEPG2 (hepatocellular carcinoma), OVCAR-3 (ovarian adenocarcinoma), SK—N—SH (neuroblastoma) and A549 (lung carcinoma). Several compounds were assayed in vitro using the Cell Titer Blue viability assay against an array of cancer cell lines of both human and murine origin plus one healthy human breast cell line (Table 1, FIGS. 1-16). A mouse 4T1 metastasis study was performed to determine the anti-metastatic potential of one compound, MBC0179 (FIG. 17).

Reagents and Equipment

Dimethyl sulfoxide (DMSO), Cremophor EL, methylene blue, 100× antibiotic/antimycotic solution, collagenase (Type IV), DNAse I and 6-thioguanine were available from Millipore-Sigma. Peptides (Compounds 1-5, Table 1) were synthesized as described above by Bio Basic, Inc. (Ontario, Canada). Peptides were dissolved in DMSO to 10 mM prior to dilution into the appropriate media. Cell Titer Blue reagent (Promega, Inc.) was used according to the manufacturer's instructions. Resorufin fluorescence was measured with the Enspire Fluorescence plate reader (PerkinElmer) using 560 nm excitation and 583 nm emission.

Cell Lines and Culture

All cell lines were procured from the American Type Culture Collection, ATCC (www.atcc.org, see Table 2 for details). Media and media supplements were available from ThermoFisher Scientific. Unless otherwise stated, cells were grown in monolayer in DMEM (Dulbecco's Modification of Eagle's Medium) media supplemented with 10% (v/v) fetal bovine serum (FBS) and 1% (v/v) antibiotic/antimycotic solution (100 units/mL penicillin, 100 μg/mL streptomycin and 0.025 μg/mL amphotericin B). hTERT-HME1 (and for one experiment, MDA-MB-231) cells were grown in MEGM (Mammary Epithelial Cell Growth Medium) media (Lonza) supplemented with 10% (v/v) FBS and 1% (v/v) antibiotic/antimycotic solution. 4T1 and OVCAR-3 cells were grown in RPMI (Roswell Park Memorial Institute) 1640 media supplemented with 10% (v/v) FBS and 1% (v/v) antibiotic/antimycotic solution. K-562 cells, a non-adherent cell line, were grown and assayed in suspension in DMEM supplemented with 10% (v/v) FBS and 1% (v/v) antibiotic/antimycotic solution. Cells were seeded in 96-well flat-bottom cell culture view plates (100 μl media per well) at 4000 cells/well and growth permitted to continue for 24 hrs at 37° C. prior to drug or vehicle incubation.

Viability Assay

Following 24 hrs of growth after seeding (Day 0), media were exchanged with that of the same composition plus vehicle (1% v/v DMSO) or Peptide (drug) in 1% v/v DMSO, added at various concentrations (as indicated on Figures and Figure legends). Drug or vehicle incubation was permitted to continue for 72 hrs at 37° C. prior to Cell Titer Blue Assay. Cell Titer Blue reagent was added to 10% v/v in each well and plates were incubated at 37° C. for 2 hrs prior to fluorescence measurement. All experimental data were the product of three biological replicates, each performed in triplicate (three technical replicates). The average baseline Resorufin fluorescence (media without cells) was subtracted from each well containing vehicle or drug with cells. Corrected fluorescence signals were then normalized to the vehicle treated wells and expressed as a percentage normalized cell viability. Data were plotted, curve-fitted to the Hill Equation and IC₅₀ values calculated using DrFit software (as described in Nature Scientific Reports 5, Article number: 14701, 2015), incorporating the average and standard error for all 3 biological replicates (FIGS. 1-16).

4T1 Mouse Breast Cancer Metastasis Study

Eight week old BALB/c female mice (3 groups of n=6 per group, vehicle plus 2 drug treatment groups) were implanted with 250000 4T1 cells in the 4^{th} inguinal mammary fat pad. One week after cell implantation, doses of 10 mg/kg (treatment, group 2) and 15 mg/kg (treatment, group 3) of VEBC0179 in carrier (7% w/v Cremophor EL and 10% ethanol in normal saline) or carrier only (control, group 1) were given by intravenous administration twice weekly. All animals were weighed and tumors were measured using calipers three times weekly. Five weeks after implantation all animals were humanely euthanized by overexposure to carbon dioxide and lungs excised for clonogenic assay according to IACUC guidelines. Both lungs of the mouse were removed, washed with Dulbecco's Phosphate buffered saline (DPBS) (VWR), minced into small pieces with scissors, broken up further by repeated pipetting through a wide bore pipette and digested in 5 mL DPBS, containing 4 mg/mL collagenase type IV and 2.5 U/mL DNase I at 37° C. for 2 hours with agitation. Samples were filtered through a 40 μm cell strainer and spun at 1000 rpm for 5 minutes, washed with 10 ml of DPBS, spun again, and finally resuspended in 5 ml of DMEM supplemented with 10% (v/v) FBS, 1% (v/v) antibiotic/antimycotic solution and 60 μM 6-thioguanine (due to resistance of 4T1 cells to 6-thioguanine, its inclusion ensures only 4T1 colonies are being counted). For each processed set of lungs (from one animal), 5 ml of concentrated cells were transferred to the first well of a 6-well cell culture plate and then diluted serially 1/10 into each of the remaining 5 wells in 5 ml of DMEM supplemented with 10% (v/v) FBS, 1% (v/v) antibiotic/antimycotic solution and 60 μM 6-thioguanine per well. The plates were incubated at 37° C. for 2 weeks prior to media removal and washing of each well twice with 5 ml of DPBS. The cells were then fixed by the addition of 1 ml of methanol for 20 minutes at room temperature. The methanol was removed and cells were incubated with 1 ml of methylene blue staining solution (0.03% w/v in water) for 10 minutes. The background methylene blue was then removed by immersing the plates in water, in order to reveal blue-stained 4T1 colonies which were subsequently counted. Taking account of the original dilution factor for each well, the total number of 4T1 colonies per set of lungs was calculated. These counts were then averaged for all subjects within each treatment or control group and standard error calculated and values plotted (FIG. 17). Finally, statistical significance of changes between control and treated groups was assessed using the two-tailed t-test.

Results

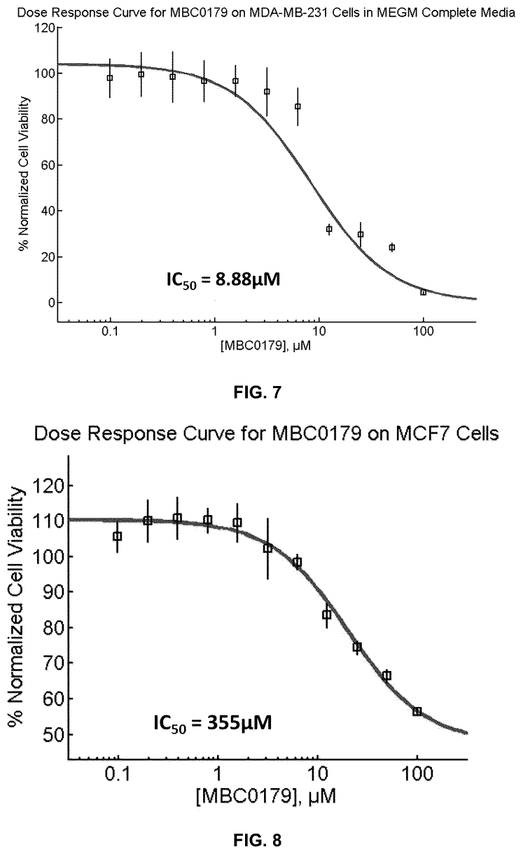

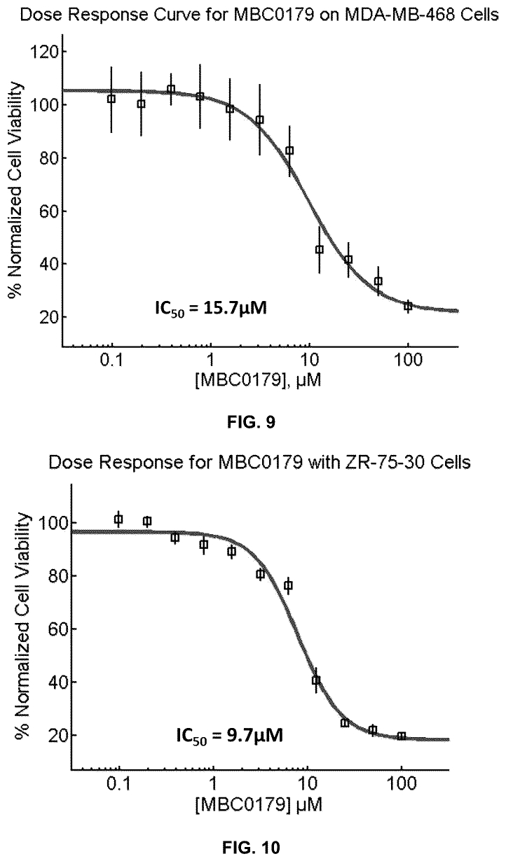

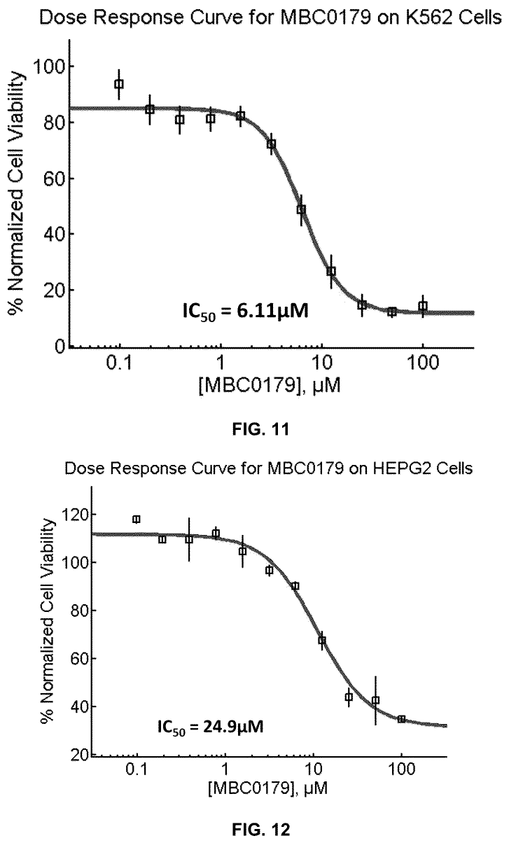

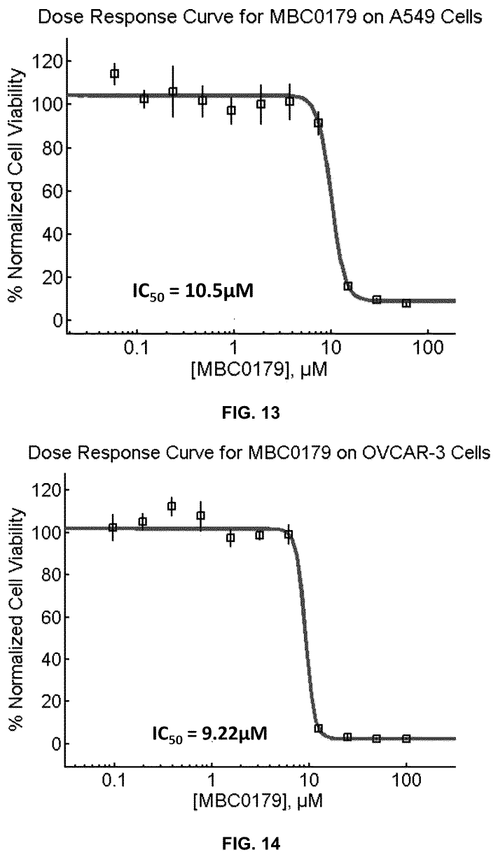

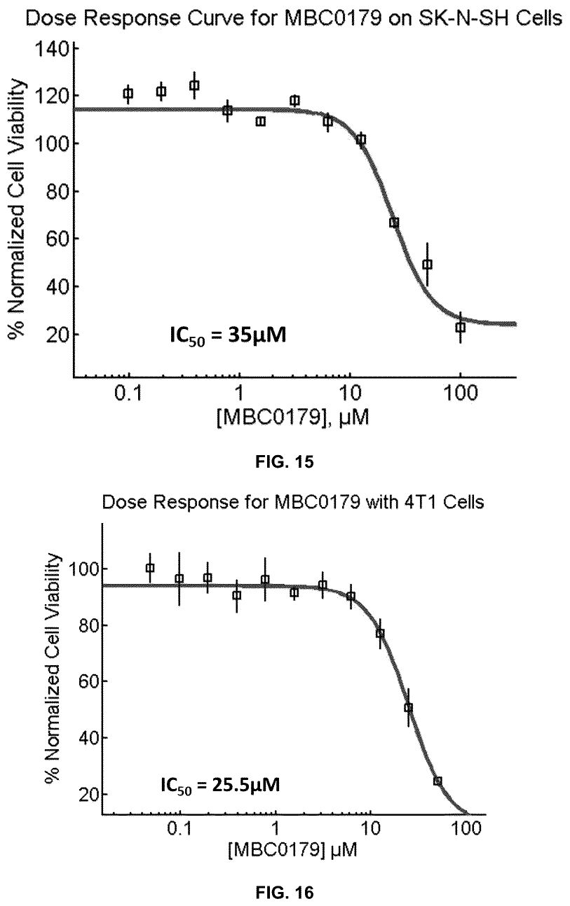

Summarized in Table 1 (and FIG. 1), N—[N-(1-oxohexadecyl)-L-histidyl]-L-valine (1) or MBC017, reduced the fraction of viable MDA-MB-231 triple-negative (Claudin-Low subtype) human breast cancer cells relative to vehicle in a dose-dependent manner after 3 days of incubation. An almost 40% reduction in viability was observed with 125 μM of MBC017, however the reduction was not sufficient to accurately calculate an IC₅₀ value. Derivatives of MBC017, namely, MBC0171, MBC0172, MBC0174 and MBC0179 also demonstrated anti-cancer effectiveness against MDA-MB-231 cells, achieving IC₅₀ values of 6730 μM, 13.1 μM, 62.1 μM and 14.7 μM, respectively (Table 1, FIGS. 2-5). hTERT-HME1, a human normal breast epithelial cell line, normally grown in growth factor-supplemented MEGM media, was tested with the compound MBC0179 resulting in a significantly increased IC₅₀ (270 μM, Table 1, FIG. 6) relative to the IC₅₀ of MBC0179 (8.88 μM, Table 1, FIG. 7) incubated in the same media with MDA-MB-231 cells. This data indicates that MBC0179 is selectively inhibitory to the growth of breast epithelial cancer cells versus normal breast epithelium. MBC0179 was further tested against other human breast cancer cell lines, namely MCF7 (ER⁺, Luminal subtype), MDA-MB-468 (triple-negative, Basal subtype), and ZR-75-30 (ER+HER2+, Luminal subtype), yielding dose dependent reductions in cell viability and resulting IC₅₀ values of 355 μM (FIG. 8), 15.7 μM (FIG. 9) and 9.7 μM (FIG. 10), respectively. These data strongly suggest that MBC0179 may be effective against a broad range of breast cancer molecular subtypes. Additionally, MBC0179 was assayed for cell viability reduction with several model human cancer cell lines representing chronic myelogenous leukemia (K562, FIG. 11, IC₅₀=6.11 μM), liver cancer (HEPG2, FIG. 12, IC50=24.9 μM), lung cancer (A549, FIG. 13, IC₅₀=10.5 μM), ovarian cancer (OVCAR-3, FIG. 14, IC₅₀=9.22 μM) and neuroblastoma (SK—N—SH, FIG. 15, IC₅₀=35 μM). These data demonstrate broad anti-cancer effectiveness, suggesting MBC0179 may be cancer agnostic. Finally, the in vitro assessment of compound MBC0179 effectiveness was completed with a dose-dependent viability experiment with the highly metastatic mouse triple negative breast cancer cell line, 4T1, in order to gain a preliminary indication of anti-metastatic potential prior to conducting a 4T1 in vivo mouse breast cancer metastasis study. The dose response data in FIG. 16 strongly indicate that the drug would be effective (IC₅₀=25.5 μM) and therefore an in vivo study was initiated (results described below).

4T1 breast cancer cells primarily metastasize from the mammary fat pads to the lungs in mice and due to the resistance of 4T1 cells to 6-thioguanine, a colonogenic assay to measure 4T1 lung metastasis has been widely adopted. Data were collected from three study groups (vehicle treated, 10 mg/kg MBC0179 treated, and 15 mg/kg MBC0179 treated), each composed of six female Balb/c mice, which were dosed twice weekly. Average 4T1 colony counts for each group were plotted (FIG. 17) and a statistically significant reduction (p=0.03, two-tailed t-test) in lung metastases was observed between the vehicle and 15 mg/kg MBC0179-treated groups. Additionally, the percent reduction in lung metastases between these two groups was 74.4% (FIG. 17). Overall, there was a dose-dependent reduction in lung metastases, indicating that MBC0179 could prevent breast cancer metastasis in vivo and may be of similar therapeutic benefit in humans.

1. A compound of formula: 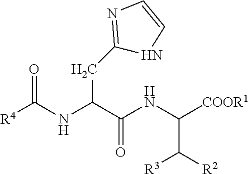
or a pharmaceutically acceptable salt thereof, wherein
R¹ is H or (C₁-C₆)alkyl;
R² is (C₁-C₁₀)hydrocarbyl;
R³ is (C₁-C₆)alkyl;
R⁴ is chosen from (C₁₄-C₂₀)hydrocarbyl; (C₁₄-C₂₀)hydrocarbyl substituted with one or more of halogen, hydroxy, mercapto, (C₁-C₆)acyl, (C₁-C₆)alkoxy, (C₁-C₆)haloalkoxy, amino, (C₁-C₆)alkylamino, di(C₁-C₆)alkylamino, and (C₁-C₆)alkylthio; (C₁₀-C₂₀)oxaalkyl; (C₁₀-C₂₀)thiaalkyl and (C₁₀-C₂₀)azaalkyl.

2. The compound of claim 1 or pharmaceutically acceptable salt thereof, wherein R¹ is H.

3. The compound of claim 1 or pharmaceutically acceptable salt thereof, wherein R² is ethyl and R³ is methyl.

4. The compound of claim 1 or pharmaceutically acceptable salt thereof, wherein R² and R³ are both methyl.

5. The compound of claim 1 or pharmaceutically acceptable salt thereof, wherein R⁴ is a (C₁₄-C₂₀)aliphatic hydrocarbon.

6. The compound of claim 5 or pharmaceutically acceptable salt thereof, wherein R⁴ is linear (C₁₄-C₂₀)alkyl.

7. The compound of claim 4 or pharmaceutically acceptable salt thereof, wherein R¹ is H and R⁴ is (C₁₄-C₂₀)alkyl.

8. The compound of claim 1 or pharmaceutically acceptable salt thereof, wherein the compound is selected from
N—[N-(1-oxohexadecyl)-L-histidyl]-L-valine;
N—[N-(1-oxohexadecyl)-L-histidyl]-L-isoleucine;
N—[N-(1-oxohexadecyl)-L-histidyl]-L-valine ethyl ester;
N—[N-(1-oxohexadecyl)-D-histidyl]-D-valine; and
N—[N-(1-oxohexadecyl)-D-histidyl]-D-valine ethyl ester.

9. A pharmaceutical composition comprising a pharmaceutically acceptable carrier and the compound of claim 1 or pharmaceutically acceptable salt thereof, or a combination of any two or more of said compounds or pharmaceutically acceptable salts thereof.

10. A method for treating breast cancer comprising administering to a subject having breast cancer a compound of claim 1 or pharmaceutically acceptable salt thereof.

11. The method of claim 10, wherein administering comprises administering two or more compounds of claim 1, pharmaceutically acceptable salts of two or more compounds of claim 1, or any combination of the foregoing.

12. A pharmaceutical composition comprising a pharmaceutically acceptable carrier and two or more compounds of claim 1, pharmaceutically acceptable salts of two or more compounds of claim 1, or any combination of the foregoing.

13. The compound of claim 3 or pharmaceutically acceptable salt thereof, wherein R⁴ is a (C₁₄-C₂₀)aliphatic hydrocarbon.

14. The compound of claim 13 or pharmaceutically acceptable salt thereof, wherein R⁴ is linear (C₁₄-C₂₀)alkyl.

15. The compound of claim 4 or pharmaceutically acceptable salt thereof, wherein R⁴ is a (C₁₄-C₂₀)aliphatic hydrocarbon.

16. The compound of claim 15 or pharmaceutically acceptable salt thereof, wherein R⁴ is linear (C₁₄-C₂₀)alkyl.

17. The pharmaceutical composition of claim 9, wherein the compound is selected from
N—[N-(1-oxohexadecyl)-L-histidyl]-L-valine;
N—[N-(1-oxohexadecyl)-L-histidyl]-L-isoleucine;
N—[N-(1-oxohexadecyl)-L-histidyl]-L-valine ethyl ester;
N—[N-(1-oxohexadecyl)-D-histidyl]-D-valine; and
N—[N-(1-oxohexadecyl)-D-histidyl]-D-valine ethyl ester.

18. The method of claim 10, wherein the compound is selected from
N—[N-(1-oxohexadecyl)-L-histidyl]-L-valine;
N—[N-(1-oxohexadecyl)-L-histidyl]-L-isoleucine;
N—[N-(1-oxohexadecyl)-L-histidyl]-L-valine ethyl ester;
N—[N-(1-oxohexadecyl)-D-histidyl]-D-valine; and
N—[N-(1-oxohexadecyl)-D-histidyl]-D-valine ethyl ester.

19. The compound of claim 13 or pharmaceutically acceptable salt thereof, wherein R¹ is H.

20. The compound of claim 15 or pharmaceutically acceptable salt thereof, wherein R¹ is H.
